(19) United States
(12) Patent Application Publication
(10) Pub. No.: US 2023/0099684 A1
(43) Pub. Date: Mar. 30, 2023

(54) DUAL-BLADE TENDON CUTTING APPARATUS

(71) Applicant: Quadvantage Technology, Inc., Raleigh, NC (US)

(72) Inventors: Paul Leach Burroughs, III, Raleigh, NC (US); Thomas Gutierrez, Macon, NC (US)

(21) Appl. No.: 17/486,257

(22) Filed: Sep. 27, 2021

(51) Int. Cl. A61B 17/3205

(52) U.S. Cl. CPC A61B 17/3205; A61B 2017/00473

(57) ABSTRACT

The present disclosure provides a dual-blade tendon cutting apparatus and cartridge for multiple apparatuses. Exemplary aspects of the present disclosure relate to a cutting implement that comprises two parallel blades that are spaced apart from one another by a predefined distance. The distance between the blades determines a lateral size of a graft being taken from the quadriceps tendon. To simplify matters for the surgeon, a cartridge is provided with three different blade sets. Much like a razor that has a disposable head, the cutting implement may attach to a desired blade set and be used.

### RELATED APPLICATIONS

The present application is related to U.S. patent application Ser. No. 16/900,315, filed Jun. 12, 2020. The '315 application claims priority to U.S. Provisional Patent Application Serial No. 62/861,623 filed on Jun. 14, 2019. The contents of both of these applications are incorporated herein by reference in their entirety.

### FIELD OF THE DISCLOSURE

The present disclosure relates to a dual-blade cutting apparatus for cutting tendons and, more particularly, for making a preliminary cut along a tendon using a dual-blade cutting apparatus.

### BACKGROUND

Most people can go through the majority of their life without ever caring or knowing how complicated a structure the knee that helps them walk is. However, the knee remains a fragile mechanical structure that is readily susceptible to damage. While medical advances have made repairing the knee possible, repair of certain types of injuries results in other long term effects. To assist the reader in appreciating the elegance of the present disclosure, FIG. 1 is provided with a brief explanation of the components of the knee.

For the purposes of the present disclosure, and as illustrated, the knee may be composed of the quadriceps muscles 100, the femur 102, the articular cartilage 104, the lateral condyle 106, the posterior cruciate ligament 108, the anterior cruciate ligament (ACL) 110, the lateral collateral ligament 112, the fibula 114, the tibia 116, the patellar tendon 118, the meniscus 120, the medial collateral ligament 122, the patella 124 (shown slightly displaced to the side—it normally rests in the center of the knee), and the quadriceps tendon 126. Of particular interest for the purposes of the present disclosure is the ACL 110 and what is done to repair the ACL 110.

ACL tears are common in athletes and are usually season-ending injuries. The ACL 110 cannot heal—it must be surgically reconstructed. The reconstruction requires replacement tissue. The most common tissue used is a central slip of the patient's own patellar tendon 118. In practice, the patellar tendon 118 has proven to be generally effective, but the size of the graft that can be used is limited to the size of the patient's own patellar tendon 118. As a rule of thumb, only a third of the patellar tendon 118 may be harvested as a graft. Thus, a doctor will measure the width of the patellar tendon 118, divide by three, and take the middle third of the patellar tendon 118. Such harvested grafts are rarely more than ten millimeters (10 mm) wide and may be smaller. Taking this tissue from a person's patellar tendon 118 also causes significant pain and discomfort in the post-operative healing period, which may last up to a year, and up to twenty (20) percent of these patients are left with chronic anterior knee pain.

Some doctors recommend and use other graft sources, such as cadaver grafts, but cadaver grafts have a higher failure rate. Additionally, there is a non-zero chance of disease transmission or rejection by the patient's immune system. As a final drawback, cadaver grafts are usually quite expensive and may not be covered by some insurance companies.

Other doctors use hamstring tendons (e.g., the distal semitendinosus tendon) because the scar created during harvesting is relatively small and there is less pain during the rehabilitation, but again, the hamstring tendon has its own collection of disadvantages. The disadvantages include the fact that once the graft is taken, a patient's hamstring will never recover to its previous strength. Further, all hamstring reconstructions stretch and are looser than the original ACL 110. This loosening is particularly problematic in younger female athletes.

Another alternative graft source is the quadriceps tendon 126. The quadriceps tendon 126 is larger and stronger than either the patellar tendon 118 or the hamstring tendon. The quadriceps tendon 126 is likewise stiffer and less prone to stretching or plastic deformation. However, the qualities that make the quadriceps tendon 126 attractive also contribute to the difficulty in harvesting a graft from the quadriceps tendon 126. Existing surgical implements require a large incision up the longitudinal axis of the femur 102 on the front or ventral/anterior side of the thigh to cut down to the level of the quadriceps tendon 126, resulting in a large post-operative scar. Additionally, the quadriceps tendon 126 has a consistency similar to the proverbial shoe leather, making it difficult to cut. However, an ACL 110 repaired with grafts from the quadriceps tendon 126 generally results in almost no anterior knee pain postoperatively over the short or long term and patients recover quicker.

U.S. Pat. Nos. 8,894,672; 8,894,675; 8,894,676; 9,044,260; 9,107,700; and 9,474,535 along with U.S. patent application Ser. No. 17/323,736 provide a number of devices designed to create a graft from the quadriceps tendon 126 as well as a number of secondary cutting implements to trim the distal end of the graft. Additionally, the '315 application introduced the concept of a dual-blade cutting device. While these devices perform admirably, there remains a desire to afford a surgeon more flexibility in approaching the preparation of a tendon for tendon harvesting.

### SUMMARY

The present disclosure provides a dual-blade tendon cutting apparatus. Exemplary aspects of the present disclosure relate to a cutting implement that comprises an attachment with two parallel blades that are adjustably spaced apart from one another and may be selectively inserted and removed from a handle device. The distance between the blades determines a lateral size of a graft being taken from the quadriceps tendon. By providing the dual blades in an adjustable and removable attachment, the surgeon has the freedom to reuse the handle portion after suitable sterilization while providing freedom to select a desired distance between blades to make a desired graft harvest. This approach reduces waste relative to the '315 cartridge approach and still provides desired cutting flexibility.

In this regard, in one aspect, a cutting assembly is disclosed. The cutting assembly comprises a handle having a longitudinal axis and a vertical axis. The handle comprises a body delimiting a receptacle along a portion of the longitudinal axis, a lock screw, and a set screw. The cutting assembly also comprises a blade attachment removably positioned in the receptacle and secured in the receptacle by the lock screw. The blade attachment comprises a stationary blade. The blade attachment also comprises a movable blade parallel to the stationary blade. The blade attachment also comprises a slider bar sandwiched between the stationary blade and the movable blade and configured to move the movable blade relative to the stationary blade based on position. The slider bar is selectively fixed in position by the set screw.

In another aspect, a blade attachment is disclosed. The blade attachment comprises a stationary blade. The blade attachment also comprises a movable blade parallel to the stationary blade. The blade attachment also comprises a slider bar sandwiched between the stationary blade and the movable blade and configured to move the movable blade relative to the stationary blade based on position.

Those skilled in the art will appreciate the scope of the disclosure and realize additional aspects thereof after reading the following detailed description in association with the accompanying drawings.

### BRIEF DESCRIPTION OF THE DRAWINGS

The accompanying drawings incorporated in and forming a part of this specification illustrate several aspects of the disclosure, and together with the description serve to explain the principles of the disclosure.

FIG. 1 illustrates a conventional knee;

FIG. 2A illustrates a perspective top view of a dual-blade cutting apparatus according to an exemplary aspect of the present disclosure;

FIG. 2B illustrates a side elevational view of the dual-blade cutting apparatus of FIG. 2A;

FIG. 2C is a top plan view of the dual-blade cutting apparatus of FIG. 2A;

FIG. 2D is a front side elevational view of the dual-blade cutting apparatus of FIG. 2A;

FIG. 3A illustrates a top plan view of a cartridge holding a plurality of dual-blade attachments suitable for use with a handle;

FIG. 3B illustrates a side elevational view of the cartridge of FIG. 3A taken along line 3B-3B of FIG. 3A;

FIG. 3C illustrates a longitudinal side elevational view of the cartridge of FIG. 3A taken along line 3C-3C of FIG. 3A;

FIG. 3D illustrates a perspective view of the cartridge of FIG. 3A with a plurality of differently-sized dual-blade attachments ready for use;

FIG. 4A illustrates a top front perspective view of a dual-blade attachment removed from the cartridge of FIG. 3A;

FIG. 4B illustrates a top plan view of the dual-blade attachment of FIG. 4A;

FIG. 4C illustrates a side elevational view of the dual-blade attachment of FIG. 4A;

FIG. 4D illustrates a rear side elevational view of the dual-blade attachment of FIG. 4A;

FIG. 5 illustrates a perspective view of a dual-blade attachment secured to a handle proximate a cartridge holding a plurality of dual-blade attachments;

FIG. 6A illustrates a cutting apparatus handle configured to receive an adjustable dual-blade cutting attachment;

FIG. 6B illustrates the cutting apparatus handle of FIG. 6A, but with an adjustable dual-blade cutting attachment inserted;

FIG. 7A provides a perspective underside view of the handle of FIG. 6A;

FIG. 7B provides a perspective underside view of the assembly of FIG. 6B;

FIG. 7C provides an alternate perspective of the handle with the dual-blade attachment removed illustrating the receptacle into which the dual-blade attachment is inserted;

FIG. 8 is an exploded perspective view of the handle of FIG. 6A and the dual-blade cutting attachment;

FIG. 9 is an exploded underside perspective view of the dual-blade cutting attachment;

FIG. 10A is a top plan view of the cutting assembly with the blades of the dual-blade cutting attachment spaced;

FIG. 10B is a top plan view of the cutting assembly with the blades of the dual-blade cutting attachment closed;

FIGS. 11A-11C illustrate movement of the pins of the sliding portion of the dual-blade cutting attachment relative to the slots of the bottom portion as the space between the blades is adjusted;

FIG. 12 shows the set screw and locking screw shown relative to the dual-blade cutting attachment; and

FIG. 13 shows a cross-sectional view of FIG. 12 illustrating how the set screw interoperates with the ridges on the sliding portion of the dual-blade cutting attachment to fix the space between the blades.

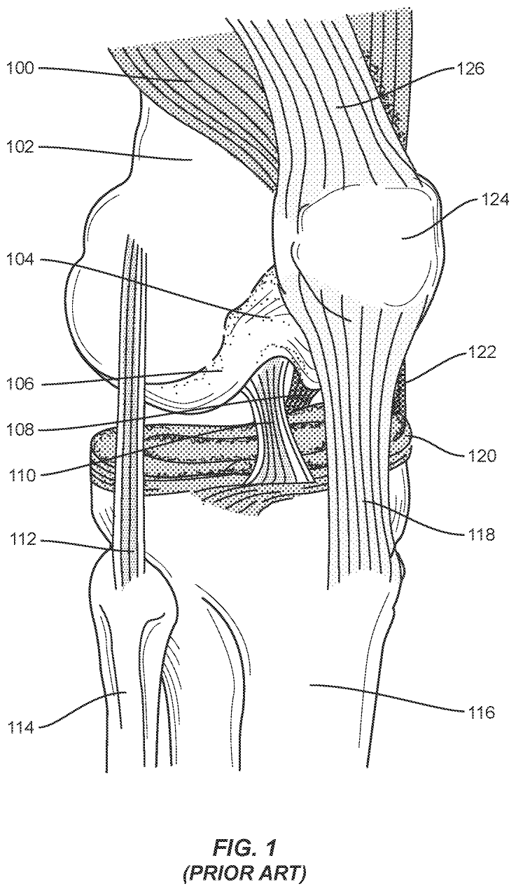

### DETAILED DESCRIPTION

The embodiments set forth below represent the necessary information to enable those skilled in the art to practice the disclosure and illustrate the best mode of practicing the disclosure. Upon reading the following description in light of the accompanying drawings, those skilled in the art will understand the concepts of the disclosure and will recognize applications of these concepts not particularly addressed herein. It should be understood that these concepts and applications fall within the scope of the disclosure and the accompanying claims.

The present disclosure provides a dual-blade tendon cutting apparatus. Exemplary aspects of the present disclosure relate to a cutting implement that comprises an attachment with two parallel blades that are adjustably spaced apart from one another and may be selectively inserted and removed from a handle device. The distance between the blades determines a lateral size of a graft being taken from the quadriceps tendon. By providing the dual blades in an adjustable and removable attachment, the surgeon has the freedom to reuse the handle portion after suitable sterilization while providing freedom to select a desired distance between blades to make a desired graft harvest. This approach reduces waste relative to the '315 cartridge approach and still provides desired cutting flexibility.

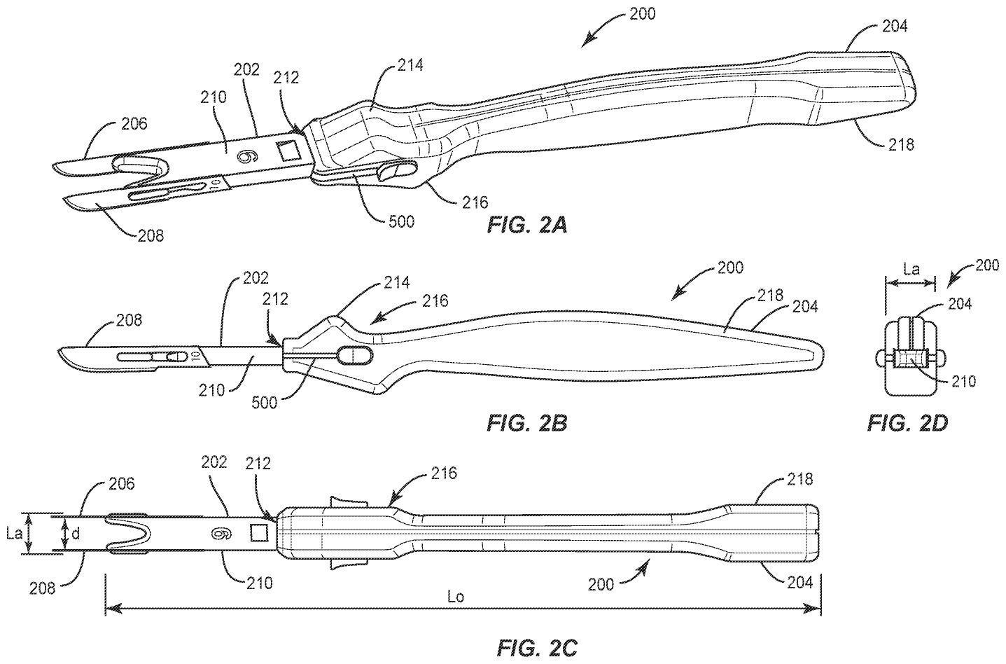

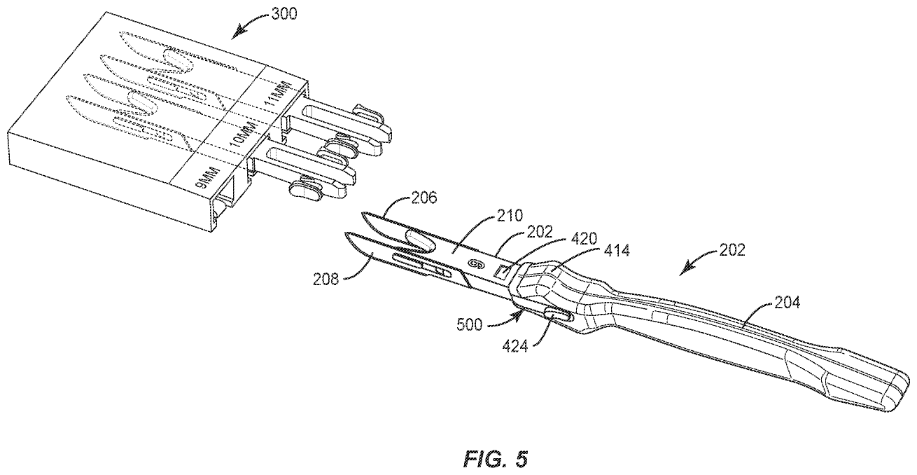

Before addressing exemplary aspects of the present disclosure, FIGS. 2A-5 are provided from the '315 application and are used to help explain some of the problem of harvesting tendons and the utility of a dual-blade cutting apparatus. A discussion of exemplary aspects of the present disclosure begins below with reference to FIG. 6A.

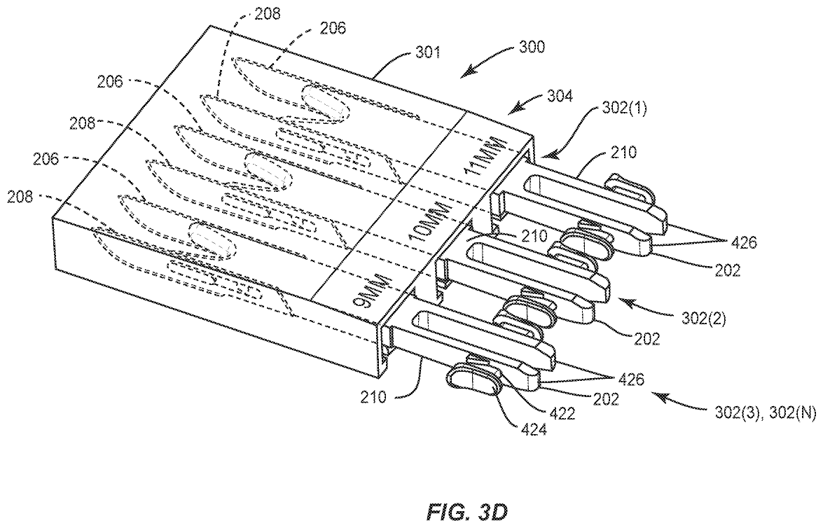

FIGS. 2A-2D illustrate a variety of views of a surgical instrument 200 that has a dual-blade cutting attachment 202 secured to a handle 204 through a snap fit arrangement. The dual-blade cutting attachment 202 has a first blade 206 in parallel with a second blade 208 along a longitudinal axis Lo, and spaced apart from one another by a distance d along a lateral axis La. The distance d corresponds to a desired dimension of a graft being harvested by the surgical instrument 200. Thus, for example, if there are nine millimeters (9 mm) between the first blade 206 and the second blade 208, a graft 9 mm wide may be harvested. The blades 206, 208 may be a metal such as surgical grade steel, although diamond, flint, or obsidian may also be used as needed or desired. The blades 206, 208 are secured to a bridge 210. The bridge 210 may be generally planar or rectilinear with a U-shaped front portion. More detail about the dual-blade attachment 202 is provided below with reference to FIGS. 4A-4D.

The handle 204 includes a channel 212 and beveled edges to allow the dual-blade cutting attachment 202 to snap fit therein. The handle 204 is contoured to provide an easy grip surface and may be made from plastic or the like. In particular, the handle 204 may include a first front ridge 214 where pressure may be applied such as by a surgeon's thumb. Likewise, the handle 204 may be generally I-shaped with a wider front section 216 and rear section 218 (best seen in FIG. 2C) to prevent or reduce the chance of a surgeon's hand slipping in the longitudinal direction Lo.

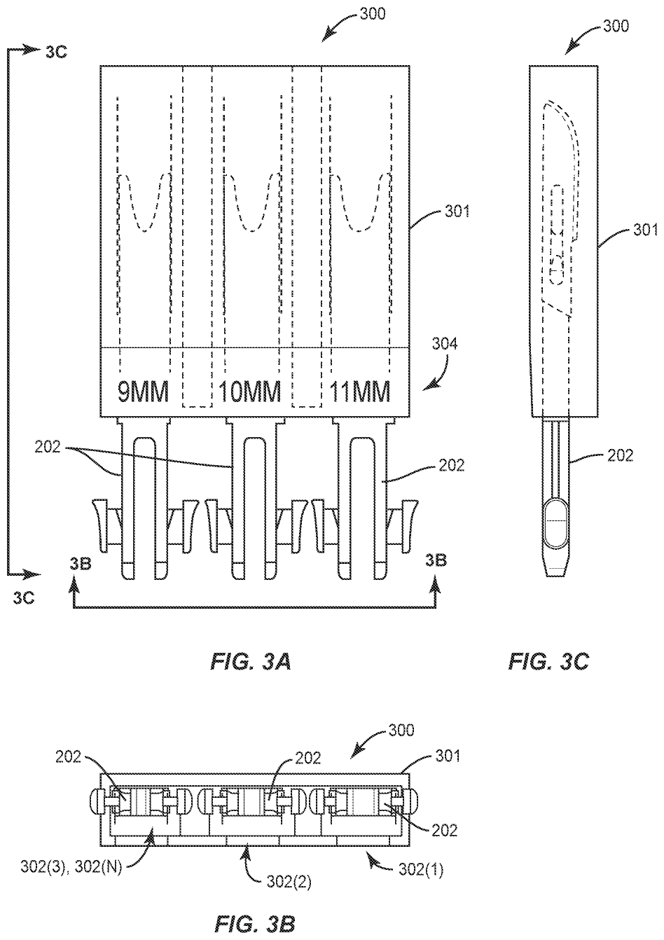

Because the size of the graft may vary depending on the person, it may be appropriate to use differently-sized dual-blade cutting attachments 202. To this end, exemplary aspects of the present disclosure allow a plurality of differently-sized dual-blade cutting attachments to be placed into respective bays of a single cartridge 300 as illustrated in FIGS. 3A-3D. In particular, the cartridge 300 may include a housing 301 which may be made of a clear or translucent plastic material and may be generally rectangular or rectilinear and may include a plurality of bays 302(1)-302(N), where as illustrated, N is three (3). In the example illustrated, widths of 9 mm, 10 mm, and 11 mm are provided for the bays 302(3)-302(1), respectively, although it should be appreciated that other widths may be provided if needed or desired. Likewise, it is possible to provide multiple dual-blade cutting attachments 202 at the same width. The housing 301 is sized so that the blades 206, 208 of the dual-blade cutting attachment 202 are within the housing of the cartridge while a portion of the bridge 210 is exposed. The housing 301 may include external indicia 304 that provide a visual indication of the distance d for the dual-blade cutting attachment 202 within a given bay 302(1)-302(N). The indicia 304 may be raised lettering or cut (e.g., through laser etching) into the housing 301.

The dual-blade cutting attachment 202 is further illustrated in FIGS. 4A-4D. In particular, the bridge 210 may include indicia 400 on an upper surface 402 that indicates the distance d between the blades 206, 208. The U-shaped portion 404 may be spaced from terminal ends 406, 408 of the blades 206, 208. The blades 206, 208 may include channels 410 that snap fit over protuberances 412, 414 of the bridge 210. The bridge 210 may further include two wing-like structures 416A, 416B that bend or compress inwardly as the handle 204 is slid over the handle end 418 of the bridge 210. A recess 420 may cooperate with a protuberance (not shown) in the housing 301 (e.g., in the bays 302(1)-302(N)) to hold the dual-blade cutting attachment 202 within a bay 302(1)-302(N) until drawn out. The wing-like structures 416A, 418 include a narrow portion 422 that fits within a channel on the handle 204 and a wide portion 424 that may be sized and contoured to fit the tip of a finger. Thus, for example, a user may use a thumb and forefinger pinching together to squeeze the wing-like structures 416A, 416B together as the narrow portion 422 is slid into the channel of the handle 204 to insert the bridge 210 into the handle 204. Once forward motion is arrested (either by the protuberance hitting the recess 420 or by terminal ends 426 hitting the end of the channel 212), the user may release the wing-like structures 416A, 416B to create a snap-fit between the bridge 210 and the handle 204.

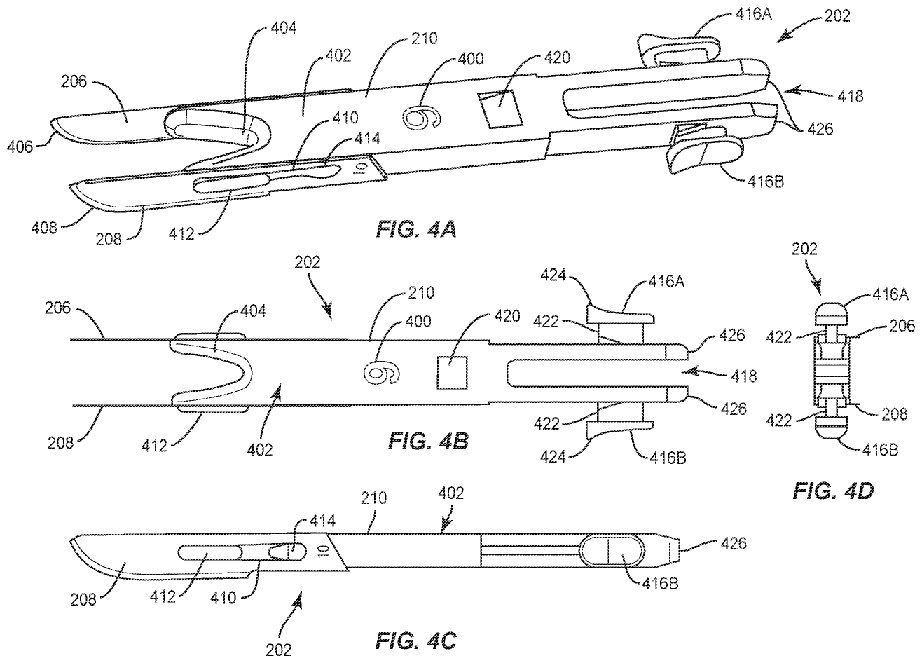

FIG. 5 illustrates the handle 204 being used to capture and extract a dual-blade cutting attachment 202 from the cartridge 300. In use, the handle 204 is grasped in one hand, and the housing 301 held in the other, and the narrow portion 422 of the bridge 210 is threaded into a channel 500 on the side of the handle 204 as the terminal ends 426 are fed into the channel 212 of the handle 204. Once threaded, the user may pinch the wing-shaped structures 416A, 416B until the dual-blade cutting attachment 202 is seated in the handle 204, then the handle 204 is pulled away from the housing 301, drawing the blades 206, 208 out of the bay 302(1)-302(N). The surgical instrument 200 is then ready for use in harvesting a tendon.

While the apparatus of the '315 application is useful in creating a graft of a desired size, it can lead to waste as only one out of three dual blades in the cartridge may be used before discarding the cartridge and the unused blades. Accordingly, exemplary aspects of the present disclosure provide an assembly formed from a handle and a removable blade attachment where the blade attachment has two parallel blades that are adjustably positioned relative to one another in such a manner as to change the lateral space between the blades.

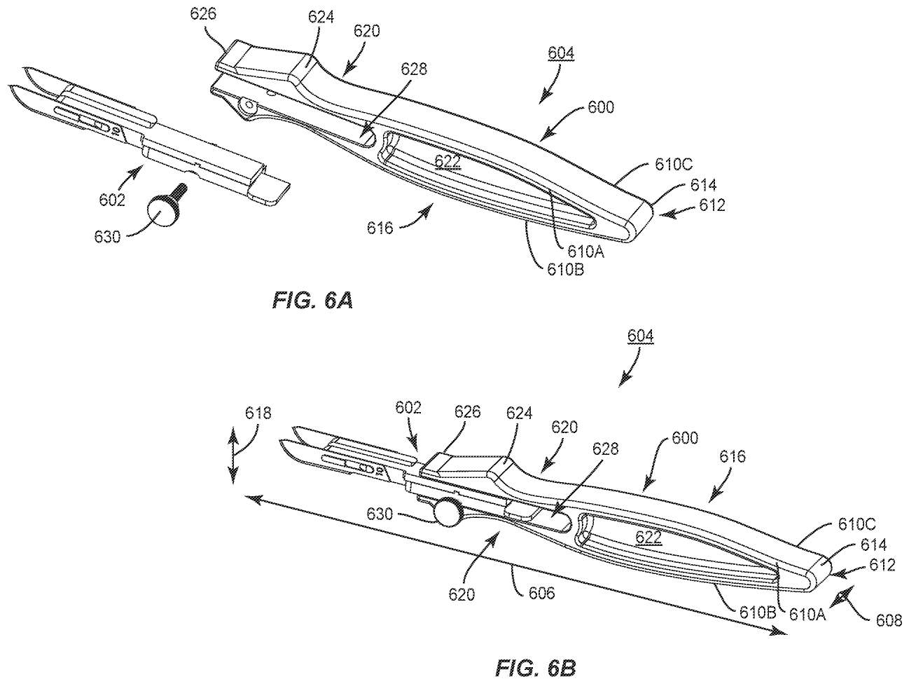

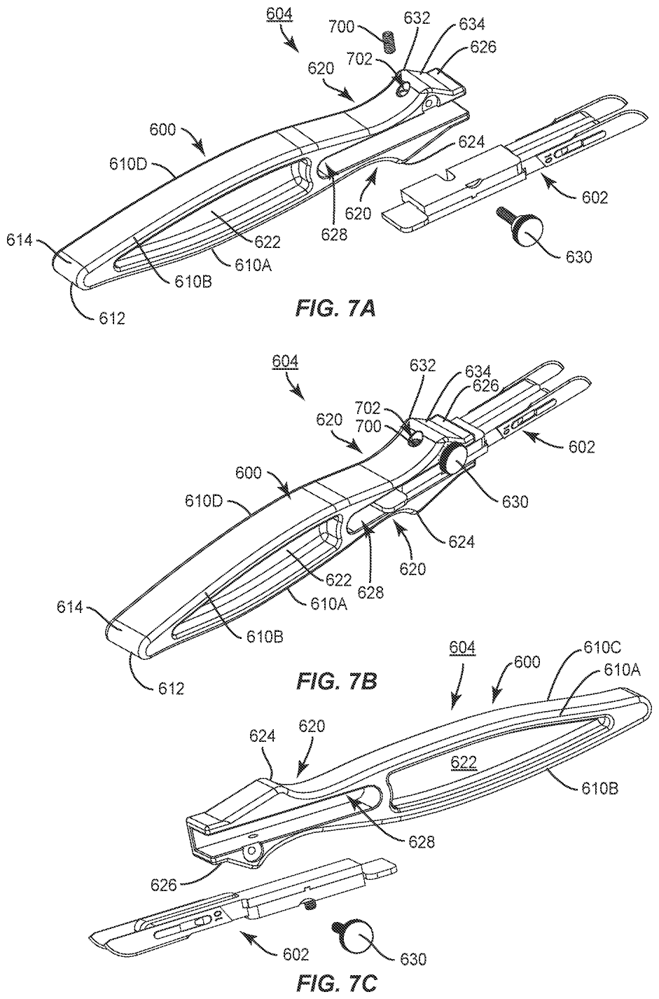

FIGS. 6A and 7A illustrate a handle 600 while FIGS. 6B and 7B illustrate the handle 600 with a blade attachment 602 installed to form an assembly 604. FIG. 7C provides alternate perspective view of the handle 600 with the blade attachment 602 removed to show the receptacle into which the blade attachment 602 is inserted. More details about the attachment 602 are provided below with reference to FIGS. 8-13.

The handle 600 has a general longitudinal axis 606 and a lateral axis 608. The handle 600 may be contoured along the lateral axis 608 with beveled edges 610A-610D (610D shown in FIGS. 7A and 7B). The handle 600 may be tapered at a first end 612 with a rounded tip 614. A middle portion 616 of the handle 600 may be bulged along a vertical axis 618 before tapering towards a waist 620. The handle 600 may delimit an interior tunnel 622 which may be provided to reduce weight and/or material expense. The interior tunnel 622 may be omitted without departing from the present disclosure. The handle 600 flares to a peak 624 in the vertical axis 618 from the waist 620 along the longitudinal axis 606 before tapering again towards a second end 626. A receptacle 628 extends from the second end 626 along the longitudinal axis 606 towards the first end 612. In an exemplary aspect, the receptacle 628 is approximately one-third of a total length of the handle 600. In general, the handle 600 is shaped to be held in an adult human hand and provide a structure that can survive a firm grip without slipping during delicate operations such as surgery. It is believed that the shape described herein and illustrated in the figures provides such a structure, but it should be appreciated that other advantages of the present disclosure may still be realized with a differently-shaped handle.

The blade attachment 602 is held in place within the receptacle 628 by a lock screw 630. It should be appreciated that the handle 600 may include a bottom swell 632 that is not necessarily directly opposite the peak 624 and has a short taper slope 634 towards the second end 626. A set screw 700 (FIG. 7A) may be inserted in a threaded passage 702 that is delimited by the bottom swell 632.

As better seen in FIG. 8, the lock screw 630 may be inserted into a threaded aperture 800. When the blade attachment 602 is placed in the receptacle 628, the lock screw 630 is threaded into the threaded aperture 800 and holds the blade attachment 602 laterally in place within the handle 600.

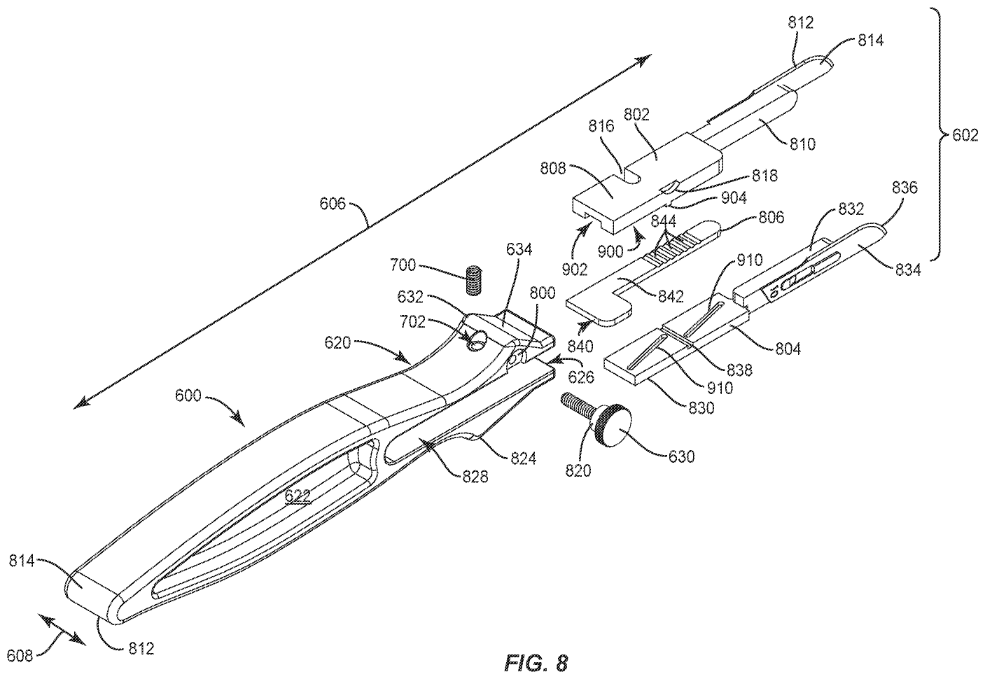

With further reference to FIGS. 8 and 9, more details about the blade attachment 602 are provided. In particular, the blade attachment 602 may include a first or stationary blade 802 and a movable blade 804. A slider bar 806 is positioned or sandwiched between the stationary blade 802 and the movable blade 804. In an exemplary aspect, the stationary blade 802 is on the bottom side of the blade attachment 602 and the movable blade 804 is positioned on the top side of the blade attachment 602. The stationary blade 802 includes a body 808 and an arm 810. The arm 810 extends from the body 808 along the longitudinal axis 606. The arm 810 has a lateral dimension less than the lateral dimension of the body 808. A first cutting blade 812 is secured to the arm 810 and extends further along the longitudinal axis 606. The first cutting blade 812 includes a cutting edge 814 shaped liked a scalpel or the like. The body 808 defines a cutout or divot 816 through which the set screw 700 may pass as better explained in greater detail below. The body 808 further includes or defines a shoulder 818 which provides a ledge on which a complementary shoulder 820 of the lock screw 630 may abut. The body 808 is generally planar on its upper surface, but as better shown in FIG. 9, a bottom surface 900 includes a channel 902 and protuberances 904.

The movable blade 804 also includes a body 830 and an arm 832. The arm 832 extends from the body 830 along the longitudinal axis 606. The arm 832 has a lateral dimension less than the lateral dimension of the body 830. A second cutting blade 834 is secured to the arm 830 and extends further along the longitudinal axis 606. The second cutting blade 834 includes a cutting edge 836 shaped liked a scalpel or the like. The body 830 defines a groove 838 which is configured to receive the protuberances 904 of the stationary blade 802. Additionally, a top surface of the body 830 may include diagonal slots 910 that interoperate with the slider bar 806 as explained below.

With continued reference to FIGS. 8 and 9, the slider bar 806 may be L-shaped with a tab 840 extending laterally from an elongated portion 842 that extends along the longitudinal axis 606. On a top surface of the slider bar 806, a series of ridges 844 may be present. The set screw 700 will extend through the handle 600, and particularly through the threaded passage 702, through the cutout 816 of the stationary blade 802 and abut the ridges 844 in such a manner that opposing ridges may trap a point end of the set screw 700 and thus prevent longitudinal movement of the slider bar 806. The bottom side of the slider bar 806 may include pegs 920 that are sized to fit within the diagonal slots 910 of the movable blade 804. While it is possible that the blade attachment 602 may be readily separable into component parts 802, 804, 806 it may also be possible to have different parts snap fit together. For example, the pegs 920 may snap fit into the diagonal slots 910 but still slide freely relative thereto and the protuberances 904 may snap fit into the groove 838, but slide freely therewithin. Note that while two pegs 920 and two diagonal slots 910 are shown, this may be modified for fewer or more of both, although at least one of each is contemplated. Likewise, there may be a single protuberance 904 without departing from the present disclosure.

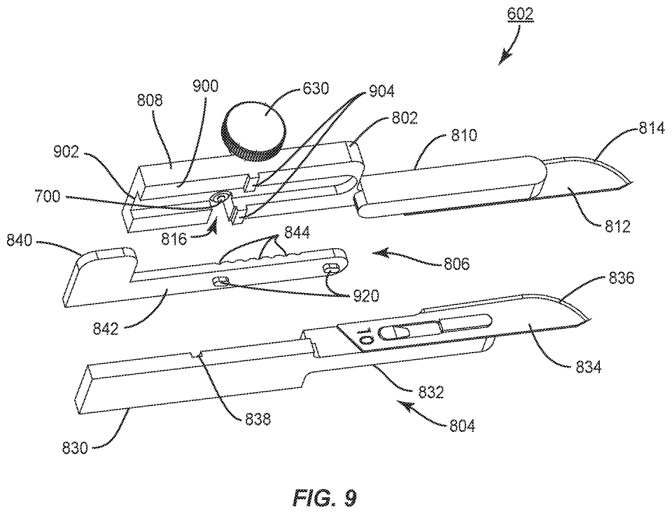

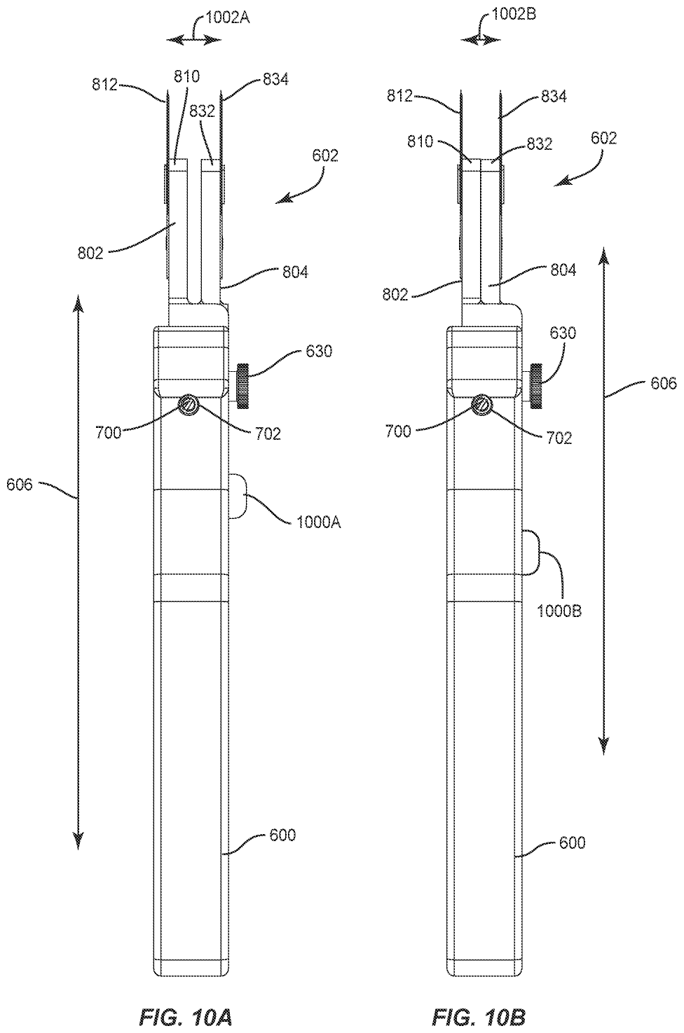

Positioning of the movable blade 804 relative to the stationary blade 802 is better illustrated in FIGS. 10A-11C. Initially, the user places the blade attachment 602 into the aperture 628 and inserts the lock screw 630 into the threaded aperture 800 to lock the blade attachment 602 laterally in place. The user then moves the tab 840 along the longitudinal axis 606 (e.g., from position 1000A to position 1000B illustrated in FIGS. 10A and 10B). The slider bar 806 will move longitudinally within the channel 902 of the stationary blade 802, but the stationary blade 802 will not move laterally. Further, moving the tab 840 in this manner causes the pegs 920 to slide within the diagonal slots 910. This movement pushes the movable blade 804 laterally such that the lateral distance between the cutting blades 812, 834 changes from width 1002A to 1002B. Once the desired width is achieved, the set screw 700 may be positioned in the threaded aperture 702 until engagement with the ridges 844 so that further longitudinal movement of the slider bar 806 is prevented. The general movement is also illustrated in FIGS. 11A-11C.

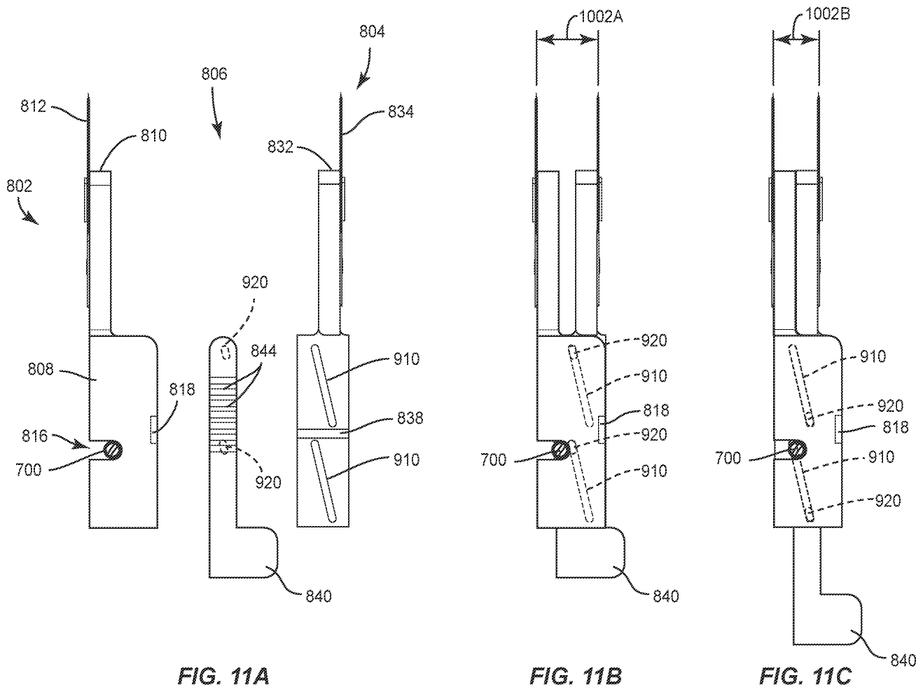

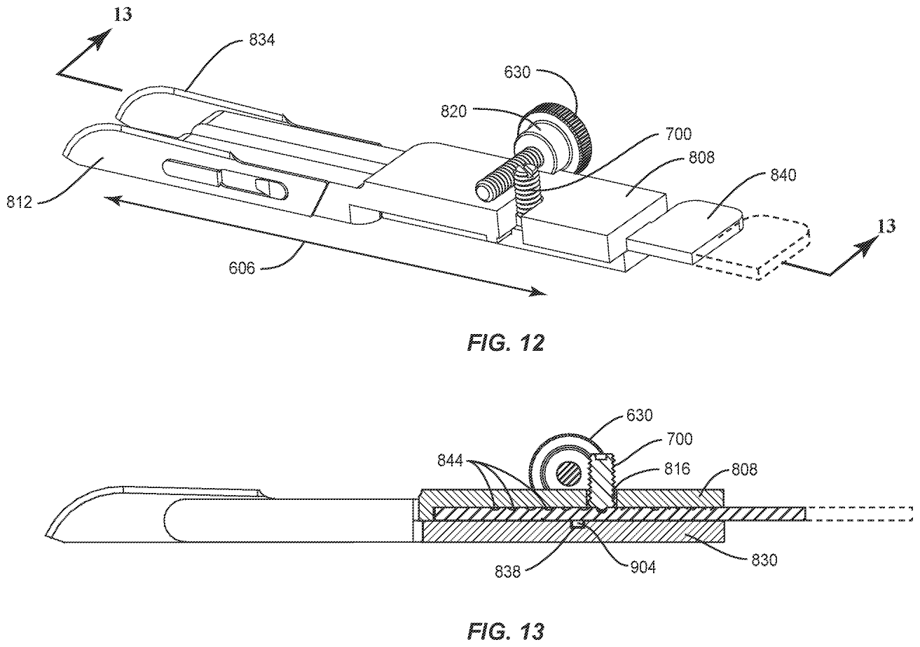

Alternate views of how the lock screw 630 and the set screw 700 inter-relate to the body 808 of the stationary blade, the body 830 of the movable blade 804, and the ridges 844 of the slider bar 806 are provided in FIGS. 12 and 13.

It should be appreciated that the handle 600 may be formed from a material that is capable of being sterilized such as in an autoclave and reused while the blade attachment 602 may be made from materials designed to be disposable or single use. Further in an exemplary aspect, the cutting blades 812 and 834 may move from between 7 mm to 14 mm width (e.g., width 1002B is 7 mm and width 1002A is 14 mm).

Given the structure of the dual-blade cutting apparatus (either using the cartridge blades of the '315 application or the attachment of the present disclosure), a brief overview of one possible use is provided. Specifically, during the preparation and harvesting of a quadriceps tendon, the surgeon may, before or after preparing a bone graft of patellar material that remains attached to the tendon, use the dual-blade cutting apparatus to “score,” “shape,” or make a preliminary guide cut in the tendon. That is, the dual-blade cutting apparatus may cut a few millimeters deep from an anterior surface of the tendon towards a posterior surface, where such cut does not cut completely from the anterior to posterior surface. These preliminary cuts on the anterior surface then help act as guides to make a consistently shaped tendon (in terms of width) for a subsequent cutting device such as the V-blade of the '672 patent or the notched blade of the '736 application to perform further cutting and harvesting. In contrast, an unguided hand may provide variations in width resulting in a suboptimal graft.

While it is specifically contemplated that the dual-blade cutting apparatus may be used in the preparation and harvesting of a quadriceps tendon, usage is not so limited and other uses including surgical (e.g., other tendons or workpieces), hobby (e.g., scoring a workpiece with parallel cuts before performing a final cut), or the like are contemplated.

Those skilled in the art will recognize improvements and modifications to the embodiments of the present disclosure. All such improvements and modifications are considered within the scope of the concepts disclosed herein and the claims that follow.

What is claimed is:

1. A cutting assembly comprising:
a handle having a longitudinal axis and a vertical axis, the handle comprising:
a body delimiting a receptacle along a portion of the longitudinal axis;
a lock screw; and
a set screw; and
a blade attachment removably positioned in the receptacle and secured in the receptacle by the lock screw, the blade attachment comprising:
a stationary blade;
a movable blade parallel to the stationary blade; and
a slider bar sandwiched between the stationary blade and the movable blade and configured to move the movable blade relative to the stationary blade based on position, the slider bar selectively fixed in position by the set screw.

2. The cutting assembly of claim 1, wherein the stationary blade comprises:
a body;
an arm extending longitudinally from the body; and
a cutting blade attached to the arm.

3. The cutting assembly of claim 1, wherein the movable blade comprises:
a body;
an arm extending longitudinally from the body; and
a cutting blade attached to the arm.

4. The cutting assembly of claim 3, wherein the movable blade further comprises a groove and at least one diagonal slot wherein the groove interoperates with a protuberance on the stationary blade and the at least one diagonal slot interoperates with a peg on the slider bar.

5. The cutting assembly of claim 1, wherein the slider bar comprises a tab and a plurality of ridges, wherein the plurality of ridges interoperate with the set screw.

6. The cutting assembly of claim 1, wherein the handle comprises a waist.

7. The cutting assembly of claim 1, wherein the handle comprises an aperture configured to receive the set screw.

8. The cutting assembly of claim 1, wherein the handle comprises a sterilizable material for reuse.

9. The cutting assembly of claim 1, wherein the movable blade comprises a first cutting blade and the stationary blade comprises a second cutting blade, and the first cutting blade may be positioned between 7 millimeters (mm) and 14 mm from the second cutting blade along a lateral axis.

10. The cutting assembly of claim 1, wherein the handle delimits an interior tunnel.

11. A blade attachment comprising:
a stationary blade;
a movable blade parallel to the stationary blade; and
a slider bar sandwiched between the stationary blade and the movable blade and configured to move the movable blade relative to the stationary blade based on position.

12. The blade attachment of claim 11, wherein the stationary blade comprises:
a body;
an arm extending longitudinally from the body; and
a cutting blade attached to the arm.

13. The blade attachment of claim 11, wherein the movable blade comprises:
a body;
an arm extending longitudinally from the body; and
a cutting blade attached to the arm.

14. The blade attachment of claim 13, wherein the movable blade further comprises a groove and at least one diagonal slot wherein the groove interoperates with a protuberance on the stationary blade and the at least one diagonal slot interoperates with a peg on the slider bar.

15. The blade attachment of claim 11, wherein the slider bar comprises a tab and a plurality of ridges, wherein the plurality of ridges interoperate with a set screw.

16. The blade attachment of claim 11, wherein the movable blade comprises a first cutting blade and the stationary blade comprises a second cutting blade, and the first cutting blade may be positioned between 7 millimeters (mm) and 14 mm from the second cutting blade along a lateral axis.
